(19) United States
(12) Patent Application Publication
(10) Pub. No.: US 2015/0258077 A1
(43) Pub. Date: Sep. 17, 2015

(54) SUBSTITUTED BENZTROPINE ANALOGS FOR TREATMENT OF DEMENTIA

(71) Applicant: P2D Inc., Cincinnati, OH (US)

(72) Inventors: Somasundar Prasad Gabbita, Loveland, OH (US); Frank Paul Zemlan, Cincinnati, OH (US)

(21) Appl. No.: 14/724,253

(22) Filed: May 28, 2015

Related U.S. Application Data
(63) Continuation of application No. PCT/US13/71810, filed on Nov. 26, 2013.
(60) Provisional application No. 61/731,634, filed on Nov. 30, 2012.

(51) Int. Cl. A61K 31/439; A61K 31/473; A61K 31/13; A61K 31/27; A61K 31/445; A61K 31/55

(52) U.S. Cl. CPC A61K 31/439; A61K 31/445; A61K 31/55; A61K 31/13; A61K 31/27; A61K 31/473

(57) ABSTRACT

A method for treating a condition selected from Alzheimer's disease, mild cognitive impairment, age-associated dementia, and frontotemporal dementia, comprises administering to a patient in need of such treatment a safe and effective amount of a compound having the formula:

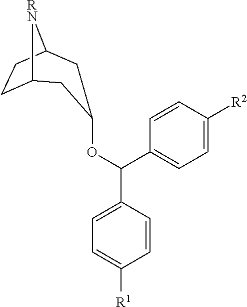

wherein R is selected from hydrogen, alkyl, alkoxy, arylalkyl, aryloxyalkyl, cinnamyl and acyl; and R¹ and R² are independently selected from hydrogen, alkyl, alkoxy, hydroxy, halogen, cyano, amino and nitro; with the proviso that if R is methyl, R¹ and R² are not both hydrogen; and the compound comprises pharmaceutically acceptable salts thereof.

### CROSS REFERENCE TO RELATED APPLICATIONS

The present application hereby claims the benefit of the provisional patent application of the same title, Ser. No. 61/731,634, filed on Nov. 30, 2013, the disclosure of which is herein incorporated by reference in its entirety.

### BACKGROUND

The brain consists of a vast network of neurons that communicate with each other via chemical messengers. Each neuron generates neurochemicals or neurotransmitters which act at sites referred to as receptors on the cellular membranes of neurons. One group of neurotransmitters, referred to as the monoamine neurotransmitters, includes serotonin, dopamine, and noradrenaline. Monoamine neurotransmitters are released into the synaptic cleft between neurons in order to stimulate post-synaptic receptor activity. The removal (or inactivation) of monoamine neurotransmitters occurs mainly by a reuptake mechanism into the presynaptic terminals. By inhibiting the reuptake, an enhancement of the physiological activity of monoamine transmitters occurs.

One monoamine neurotransmitter, the dopamine neural system of the brain, has been shown to influence a variety of physiologic functions, and compounds inhibiting reuptake by inhibiting dopamine transporter activity (dopamine transport inhibitors) have been shown to have the ability to treat in mammals, including humans, a variety of disorders associated with this neural system, for example, eating disorders, depression, cocaine addiction and attention deficit hyperactivity disorder.

However, the use of dopamine transport inhibitors to treat such conditions frequently brings along with it a number of undesirable side effects. For example, benztropine (COGENTIN) is a high affinity dopamine transport (DAT) inhibitor that increases dopamine activity in the brain. This material has been in continuous clinical use for over forty years. Benztropine's inhibition of the dopamine transporter is responsible for its clinical effectiveness for treating idiopathic Parkinson's disease, a clinical indication for which it is FDA approved. Unfortunately, the clinical usefulness of benztropine has been severely limited by its anticholinergic properties which result from benztropine's high affinity binding to M1 cholinergic receptors. Benztropine's anticholinergic side effects, as documented in the Physician's Desk Reference, include tachycardia, constipation, vomiting, confusion, disorientation, memory impairment and hallucinations.

The compounds described herein are benztropine analogs that provide the therapeutic benefits of benzatropine; however, they demonstrate reduced side effects including reduced anticholinergic side effects due to limited Ml cholinergic binding; thereby demonstrating a clinically enhanced safety profile.

U.S. Pat. No. 5,792,775, Newman, et al., issued Aug. 11, 1998, describes the family of 4′,4″-substituted 3α-(diphenylmethoxy) tropane analogs described herein and teaches their use for the treatment of cocaine addiction and for the diagnosis and/or monitoring (but not the treatment of) neurodegenerative disorders, such as Parkinson's disease.

### BRIEF SUMMARY

A method for treating a condition selected from Alzheimer's disease, mild cognitive impairment, age-associated dementia, and frontotemporal dementia, comprises administering to a patient in need of such treatment a safe and effective amount of a compound having the formula:

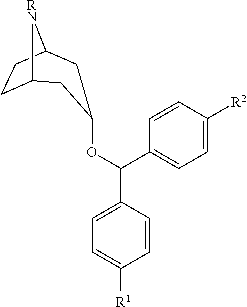

wherein R is selected from hydrogen, alkyl, alkoxy, arylalkyl, aryloxyalkyl, cinnamyl and acyl; and R¹ and R² are independently selected from hydrogen, alkyl, alkoxy, hydroxy, halogen, cyano, amino and nitro; with the proviso that if R is methyl, R¹ and R² are not both hydrogen; and the compound comprises pharmaceutically acceptable salts thereof.

### BRIEF DESCRIPTION OF THE FIGURES

The accompanying drawing, which is incorporated in and constitute a part of this specification, illustrate embodiments, and together with the general description given above, and the detailed description of the embodiments given below, serve to explain the principles of the present disclosure.

FIG. 1 is a chart of the improvement of memory of 3×Tg-AD mice treated with PD2005 compared to a control.

### DETAILED DESCRIPTION

The compounds herein are described in U.S. Pat. No. 5,792,775, Newman, et al., issued Aug. 11, 1998, incorporated herein by reference in its entirety, as well as pharmaceutically acceptable salts of those compounds. The method of making those compounds is also described in the Newman, et al. patent. These referenced 4′,4″-substituted 3α-(diphenylmethoxy) tropane analogs demonstrate high affinity for the dopamine transporter and inhibit dopamine uptake, while also exhibiting relatively limited Ml cholinergic binding. One compound for use in treating diseases is N-allyl-4′,4″-difluoro-3α-diphenyl-methoxytropane (PD2005). Another compound for use in treating diseases is N-butyl-4′,4″-difluoro-3α-diphenyl-methoxytropane (PD2007).

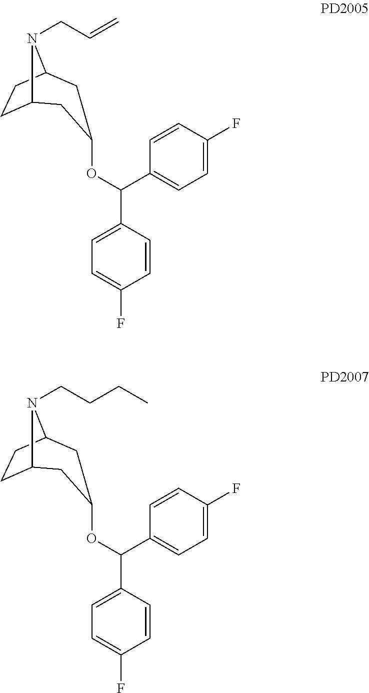 [SMILES: C=CCN1C2CCC1CC(OC(c1ccc(F)cc1)c1ccc(F)cc1)C2 ; CCCCN1C2CCC1CC(OC(c1ccc(F)cc1)c1ccc(F)cc1)C2]

The methods of treating Alzheimer's disease, mild cognitive impairment, age-associated dementia, and frontotemporal dementia uses compounds having the following formula:

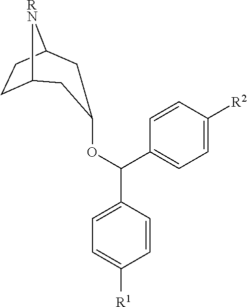

wherein R is a functional group including, but not limited to, hydrogen, alkyl, alkoxy, arylalkyl, aryloxyalkyl, cinnamyl and acyl. R¹ and R² are independently selected and are functional groups including, but not limited to, hydrogen, alkyl, alkoxy, hydroxy, halogen, cyano, amino and nitro. In these compounds, when R is methyl, R¹ and R² cannot both be hydrogen. The compound additionally comprises pharmaceutically acceptable salts thereof.

The term “independently selected” is used herein to indicate that the R¹ and R² groups can be identical or different (e.g., R¹ and R² may both be methoxy, or R¹ may be methoxy and R² may be halogen).

The term “alkyl” is used herein to refer to a branched or unbranched, saturated or unsaturated, monovalent hydrocarbon radical containing from 1-8 carbons, cycloalkyls (3-7 carbons), cycloalkyl methyls (3-8 carbons) and arylalkyls. Suitable alkyl radicals include, for example, methyl, ethyl, n-propyl, i-propyl, 2-propenyl (or allyl), n-butyl, t-butyl, i-butyl (or 2-methylpropyl), cyclopropylmethyl, i-amyl, n-amyl, hexyl, etc. As used herein, the term “alkyl” encompasses “substituted alkyl.” The term “substituted alkyl” refers to alkyls as just described above including one or more functional groups such as lower alkyl, aryl, aralkyl, acyl, halogen (i.e., haloalkyls, e.g., CF₃), hydroxyl, amino, acylamino, acyloxy, alkoxyl, mercapto, and the like. These groups may be attached to any carbon atom in the alkyl moiety.

The term “alkoxy” is used herein to refer to the —OR group, where R is a lower alkyl, substituted lower alkyl, aryl, substituted aryl, aralkyl, or substituted aralkyl. Suitable alkoxy radicals include, for example, methoxy, ethoxy, phenoxy, t-butoxy, etc.

The term “aryl” refers to an aromatic substituent which may be a single ring or multiple rings which are fused together, linked covalently, or linked to a common group such as an ethylene or methylene moiety. The aromatic ring(s) may include phenyl, naphthyl, biphenyl, biphenylmethyl, 2,2-diphenyl-1-ethyl, and may contain a heteroatom, such as thienyl, pyridyl, and quinoxalyl. The aryl group may also be substituted with halogen atoms or other groups, such as nitro, carboxy, alkoxy, phenoxy, and the like. Additionally, the aryl group may be attached to other moieties at any position on the aryl radical which would otherwise be occupied by a hydrogen atom (such as 2-pyridyl, 3-pyridyl, and 4-pyridyl). As such, the terms “aralkyl” and “aryloxyalkyl” refer to an aryl radical attached directly to an alkyl group (e.g., 3(2-pyridyl)propyl)) or an oxygen which is attached to an alkyl group, respectively.

The term “cinnamyl” is used herein to refer to the 3-phenyl-2-propenyl radical (i.e., Ph-CH═—CH₂—). The phenyl group may be substituted with halogen atoms or other groups (e.g., nitro, hydroxy, amino, etc.).

The term “acyl” is used herein to refer to the group —C(O)R, where R is hydrogen, alkyl, substituted alkyl, aryl, or substituted aryl, as defined above.

The term “cyano” is used herein to refer to the group —CN.

The term “halogen” is used herein to refer to fluorine, bromine, chlorine, and iodine atoms.

The term “hydroxyl” is used herein to refer to the group —OH.

The term “nitro” is used herein to refer to the group —NO₂.

The term “amino” is used herein to refer to the group —NRR′, where R and R′ may independently be hydrogen, lower alkyl, substituted lower alkyl, aryl, substituted aryl or acyl.

In some embodiments R is methyl; R¹ is methoxy; and R² is selected from H and methoxy. In some embodiments R is methyl; R¹ is nitro; and R² is H. In some embodiments R is methyl; R¹ is cyano; and R² is H. In some embodiments R is methyl; R¹ is Br; and R² is selected from H, Br, CI and F. In some embodiments R is methyl; R¹ is F; and R² is selected from H, Br, F and CI.

In some embodiments R is methyl; R¹ is an alkyl selected from methyl, ethyl, propyl, butyl, i-butyl, t-butyl, pentyl, and hexyl; and R² is selected from H and alkyl. In some embodiments R is methyl; R¹ is hydroxy; and R² is selected from H, hydroxy, Br, CI, and F. In some embodiments R is alkyl; and R¹ and R² are independently selected from Br, CI, F, and I. In some embodiments R is n-cinnamyl; and R¹ and R² are independently selected from Br, CI, F, and I. In some embodiments R is arylalkyl; and R¹ and R² are independently selected from Br, CI, F, and I. In some embodiments R is methyl and both R¹ and R² are fluorine atoms. Structures of such specific compounds are shown below:

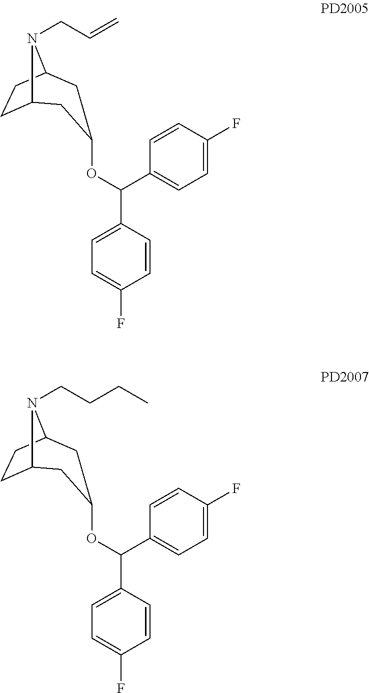 [SMILES: C=CCN1C2CCC1CC(OC(c1ccc(F)cc1)c1ccc(F)cc1)C2 ; CCCCN1C2CCC1CC(OC(c1ccc(F)cc1)c1ccc(F)cc1)C2]

Compound PD2005 has R=Allyl, and R¹═R²═F. Compound PD2007 has R=n-butyl and R¹═R²═F.

The compounds of Formula I can be prepared using the synthetic scheme set forth in the Newman, et al. patent (U.S. Pat. No. 5,792,775). Briefly, 4′,4″-substituted benzhydrols are converted to benzhydrochlorides in refluxing thionyl chloride. Benzhydrochlorides are then added, neat or in a minimal volume of anhydrous diethyl ether, to tropine at 160° C., to form 4′ or 4′,4″-substituted 3α-(diphenylmethoxy) tropane analogs. This second step, i.e., the melt reaction, can be carried out rapidly and without the use, or alternatively with the minimal use, of solvent.

The compounds described above are administered to a patient having a condition selected from Alzheimer's disease, mild cognitive impairment, age-associated dementia, and frontotemporal dementia. A primary focus is the treatment of cognitive dysfunction related to Alzheimer's disease and frontotemporal dementia. The compounds described above not only treat those conditions, but also, because of their decreased affinity for the Ml receptor, are accompanied by minimized anticholinergic side effects.

Alzheimer's disease is divided into familial and sporadic forms, with more than 20 million cases worldwide. Alzheimer's disease is considered familial when more than one person in a family is affected, while sporadic refers to cases when no other cases have been seen in close family members. Approximately 25% of Alzheimer's disease is familial, with the rest being sporadic. Alzheimer's disease is further divided into early and late-onset forms; early-onset denotes onset of the disease before age 65 years, while late-onset denotes onset after age 65 years. Almost all cases of sporadic Alzheimer's disease are late-onset, while approximately 90% of familial Alzheimer's disease is early-onset. Less than 10% of all Alzheimer's disease cases are familial early-onset.

Alzheimer's disease is a complex disease, and a number of genes have been discovered that may increase the risk of developing the disease. The most well established link between Alzheimer's disease and genetics is in familial early-onset Alzheimer's disease. Three genes have been identified that account for a significant number of familial early-onset Alzheimer's disease cases. The APP (amyloid precursor protein) gene encodes the Amyloid Precursor Protein, which is normally cleaved to form amyloid β. Mutations in APP result in incorrect cleavage of the protein, producing a version of amyloid β that is more likely to form plaques. Mutations in APP account for 10%-15% of familial early-onset cases. The PSEN (presenilin) genes encode proteins that function in the cleavage of Amyloid Precursor Protein. Mutations in both PSEN1 and PSEN2 result in incorrect cleavage of APP, and are associated with development of familial early-onset Alzheimer's disease. Mutations in PSEN1 are thought to account for 30%-70% of familial early-onset cases, while mutations in PSEN2 are thought to account for less than 5%. Familial early-onset Alzheimer's disease is inherited in an autosomal dominant manner, meaning that inheritance of one mutant allele of APP, PSEN1, or PSEN2 almost always results in development of the disease.

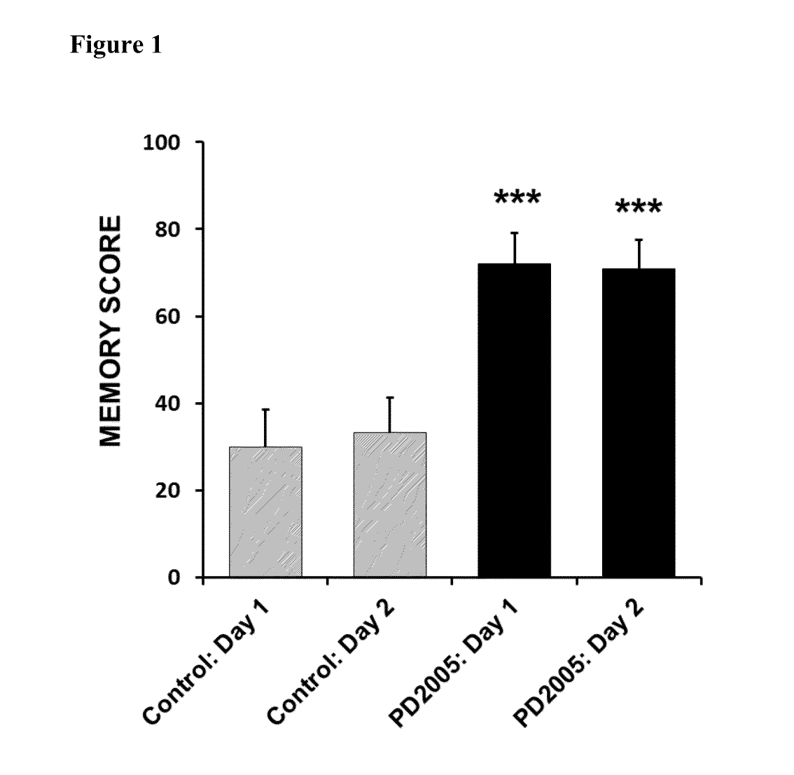

Dopamine transport inhibitors have been used to treat patients with dementia (Alzheimer's disease, frontotemporal dementia, and vascular dementia). (Dolder, C. R., et al., Use of psychostimulants in patients with dementia. The Annals of Pharmacotherapy, 2010; 39:1624-32). In these studies two different types of widely prescribed dopamine transporter inhibitors were evaluated: 1) methylphenidate (e.g., Ritalin™ or Concerta™), and 2) amphetamine (e.g., Adderall™ or Vyvanse™). When used to treat patients with dementia, these two dopamine transport inhibitors were not effective. A recent published review of these studies by a well-known physician concluded that these dopamine transport inhibitors “do not appear to be broadly effective treatments for behavioral or cognitive symptoms of dementia.” (Dolder, C. R., et al., Use of psychostimulants in patients with dementia. The Annals of Pharmacotherapy, 2010; 39:1624-32). Further, this author states that published studies do not endorse the use of dopamine transport inhibitors “as cognitive enhancers in patients with dementia.” Therefore, it is clear that dopamine transport inhibitors are not recommended treatments for dementia. In contrast, it has been surprisingly found that the claimed compounds have been shown to be highly effective at improving memory in a preclinical dementia model (FIG. 1).

The compounds described above may be administered by any conventional route, such as orally, transdermally, subcutaneously, parenterally, intramuscularly, intravenously, intraperitoneally, or via inhalation. Oral, parenteral, and subcutaneous administration are preferred. The compounds described above may be administered alone or in combination with other therapies conventionally known for treating Alzheimer's disease, mild cognitive impairment, age-associated dementia, and frontotemporal dementia.

The active compounds are administered to a patient in a “safe and effective amount,” i.e., an amount which provides the desired clinical benefit based on size, weight, age, physical and mental condition of the patient, and severity of the condition being treated, while minimizing any undesirable side effects. The precise dosages to be administered will be determined based on the judgment of the treating physician. Typical dosages for administration of the active compounds are from about 0.05 to about 1000 milligrams (mg) per day, such as from about 0.1 to about 100 mg per day, from about 0.1 to about 75 mg/day, from about 0.1 to about 50 mg/day, or from about 5 to about 10 mg/day. The desired dosage may be administered in one, two or three subdoses at suitable times during the day. The subdoses may consist of 0.05 to 1000 mg per subdose, such as 0.1 to 100 mg per subdose, or 0.5 to 10 mg per subdose. The desired dosage will depend on the particular compound to be utilized, the disease to be treated, the severity of the disease, the route of administration, the weight and health of the patient, and the judgment of the treating physician. The active compound may be administered in a timed or delayed release dosage form thereby allowing treatment over an extended period of time.

For oral administration, conventional solid carriers for the active compound may be employed, such as pharmaceutical grades of cellulose, glucose, lactose, mannitol, magnesium stearate, sodium saccharin, sucrose, talcum or similar solid carriers. A pharmaceutically acceptable dosage for oral administration may be manufactured incorporating any customary nontoxic pharmaceutical excipient, such as those excipients described above, and generally about 5% to about 95% of the active compound, such as about 25% to 75% of the active compound.

The compounds described herein, together with a conventional pharmaceutically acceptable adjuvant, carrier or diluent, may thus be placed into the form of a pharmaceutical composition and unit dosages thereof, and in such form may be employed as solids, such as tablets or filled capsules, or liquids, such as solutions, suspensions, emulsions, elixirs or capsules filled with the same, all for oral use; in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The compounds described herein can be administered in a wide variety of oral and parenteral dosage forms. It will be obvious to those skilled in the art that the following dosage forms may comprise, as the active component, either a compound described herein or a pharmaceutically acceptable salt of such a compound.

For preparing pharmaceutical compositions of the compounds described herein, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid admixed with the finely divided active component.

In tablets, the active component is mixed with the carrier having the necessary binding capacity in suitable proportions and compacted in the shape and size desired.

The powders and tablets typically contain from about 5 or 10% to about 70% of the active compound. Suitable carriers include, for example, magnesium carbonate, magnesium stearate, talc, sugar, lactose, pectin, dextrin, starch, gelatin, tragacanth, methylcellulose, sodium carboxymethylcellulose, low melting wax, cocoa butter, and the like. The description herein is intended to include the formulation of the active compound with an encapsulating material as carrier providing a capsule in which the active compound, with or without additional carriers, is surrounded by the encapsulating material, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active material is dispersed homogeneously therein, as by stirring. The molten homogeneous mixture is then poured into convenient sized molds, allowed to cool, and thereby to solidify.

Formulations suitable for vaginal administration may be presented as pessaries, tampons, creams, gels, pastes, foams or sprays containing, in addition to the active ingredient, such carriers as are known in the art to be appropriate.

Liquid form preparations include solutions, suspensions, and emulsions, for example, water or water-propylene glycol solutions. Parenteral injection liquid preparations can be formulated as solutions in, for example, aqueous polyethylene glycol solution.

The compounds described above may be formulated for parenteral administration (e.g., by injection, for example, bolus injection or continuous infusion) and may be presented in unit dosage form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active compound in water and adding suitable colorants, flavors, preservatives, stabilizing and/or thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

In some embodiments, the method comprises using solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis, the compounds described above may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oil base and will in general also contain one or more art-known emulsifying agents, stabilizing agents, dispersing agents, suspending agents, thickening agents, or coloring agents.

Formulations suitable for topical administration in the mouth include lozenges comprising active component in a flavored base, usually sucrose and acacia or tragacanth; pastilles, comprising the active component in an inert base such as gelatin or glycerin; and mouthwashes comprising the active component in a suitable liquid carrier.

Solutions or suspensions are applied directly to the nasal cavity by conventional means, for example, with a dropper, pipette or spray. The formulations may be provided in single or multidose form. In the case of a dropper or pipette, this may be achieved by the patient administering an appropriate, predetermined volume of the solution or suspension. In the case of a spray, the active component may be administered, for example, by means of a metering atomizing spray pump.

Administration to the respiratory tract may also be achieved by means of an aerosol formulation in which the active component is provided in a pressurized pack with a suitable propellant, such as a chlorofluorocarbon, for example, dichlorodifluoromethane, trichlorofluoromethane, or dichlorotetrafluoroethane, carbon dioxide, or other suitable gas. The aerosol may conveniently also contain a surfactant, such as lecithin. The dosage of drug may be controlled by provision of a metered valve.

Alternatively, for nasal administration, the active ingredient may be provided in the form of a dry powder, for example, a powder mix of the therapeutic compound in a suitable powder base, such as lactose, starch, starch derivatives such as hydroxypropylmethyl cellulose and polyvinylpyrrolidone (PVP). Conveniently, the powder carrier will form a gel in the nasal cavity. The powder composition may be presented in unit dosage form, for example, in capsules or cartridges of, e.g., gelatin, or blister packs from which the powder may be administered by means of an inhaler.

In formulations intended for administration to the respiratory tract, including intranasal formulations, the compound will generally have a small particle size, for example of the order of 5 microns or less. Such a particle size may be obtained by means known in the art, for example by micronization.

When desired, formulations adapted to give sustained release, timed release or delayed release of the active component may be employed.

The pharmaceutical preparations are preferably in unit dosage forms. In such form, the preparation is subdivided into unit doses containing appropriate quantities of the active component. The unit dosage form can be packaged, the package containing discrete quantities of preparation, such as a packeted tablet, capsule or powders in a vial or ampoule. Also, the unit dosage form can be a capsule, tablet, cachet, or lozenge itself, or it can be the appropriate number of any of these in packaged form.

Tablets or capsules for oral administration and liquids for intravenous administration are preferred pharmaceutical compositions for use in the method described herein.

All percentages, proportions and ratios set forth herein are “by weight,” unless otherwise specified.

While the present disclosure has illustrated by description several embodiments and while the illustrative embodiments have been described in considerable detail, it is not the intention of the applicant to restrict or in any way limit the scope of the appended claims to such detail. Additional advantages and modifications may readily appear to those skilled in the art.

### Example

PD2005 has been shown to be highly effective in treating Alzheimer's disease in a well-respected preclinical model. In this model, genes that cause Alzheimer's disease have been cloned from living Alzheimer's patients and then directly cloned into the genome of living mice to create Alzheimer mice (3×Tg-AD mice). These 3×Tg-AD mice demonstrate: 1) the age-dependent memory deficits seen in Alzheimer's patients, 2) the age-dependent development of brain pathology seen in Alzheimer's patients, and 3) the age-dependent dementia characteristic of Alzheimer's patients. (Oddo, S., et al., Triple-transgenic model of Alzheimer's disease with plaques and tangles: intracellular Abeta and synaptic dysfunction. Neuron, 2003; 39(3):409-21.)

PD2005 administration significantly improved memory, the central cognitive deficit seen in Alzheimer's patients, in these Alzheimer 3×Tg-AD mice (FIG. 1). Short-term memory was assessed in a well-accepted memory model (single alternation T-maze). In these studies, animals were administered 10 mg/kg PD2005 in saline 1 hr prior to testing and controls were administered only saline. PD2005 treatment caused a highly significant 140% improvement in memory as soon as the first day of testing with a highly significant 113% memory improvement also seen on the following day of testing (***p's<0.001 both days compared to controls). These data indicate that PD2005 improves the core symptom of Alzheimer's disease, memory, which was caused in these mice by the same genes that causes this disease in Alzheimer's patients.

1. A method for treating a condition selected from Alzheimer's disease, mild cognitive impairment, age-associated dementia, and frontotemporal dementia, comprising administering to a patient in need of such treatment a safe and effective amount of a compound, having the formula: 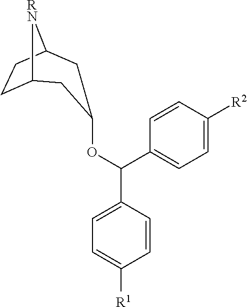
wherein R is selected from hydrogen, alkyl, alkoxy, arylalkyl, aryloxyalkyl, cinnamyl, and acyl; and
R¹ and R² are independently selected from hydrogen, alkyl, alkoxy, hydroxy, halogen, cyano, amino and nitro;
with the proviso that if R is methyl, R¹ and R² are not both hydrogen; and the compound comprises pharmaceutically acceptable salts thereof.

2. The method according to claim 1, wherein the compound is administered to the patient at from about 0.5 to about 1000 mg per day.

3. The method according to claim 1, wherein the compound is administered to the patient at from about 0.1 to about 100 mg per day.

4. The method according to claim 1, wherein R is methyl, and R¹ and R² are both fluorine.

5. The method according to claim 1, wherein R is methyl, and R¹ and R² are both chlorine.

6. The method according to claim 1, wherein R is alkyl, and R¹ and R² are selected from hydrogen and halogen.

7. The method according to claim 6 wherein R¹ is bromine and R² is selected from hydrogen, bromine, chlorine and fluorine.

8. The method according to claim 1, wherein the compound is administered as part of a pharmaceutical composition together with a pharmaceutically acceptable carrier.

9. The method according to claim 1, wherein the condition is familial Alzheimer's disease.

10. The method according to claim 1, wherein the compound is administered to the patient orally.

11. The method according to claim 1, wherein the compound is administered to the patient together with a conventional therapy for the condition being treated.

12. The method according to claim 1, wherein the dosage is from about 5 to about 10 mg per day.

13. The method according to claim 1, wherein the compound is:

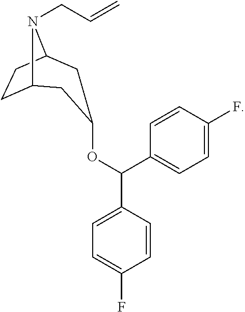 [SMILES: C=CCN1C2CCC1CC(OC(c1ccc(F)cc1)c1ccc(F)cc1)C2]

14. The method according to claim 1, wherein the compound is:

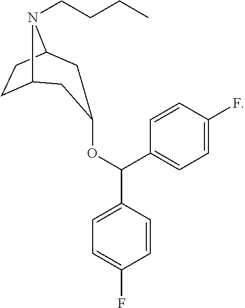 [SMILES: CCCCN1C2CCC1CC(OC(c1ccc(F)cc1)c1ccc(F)cc1)C2]

15. The method according to claim 1, wherein the compound is administered to the patient together with one or more of the following: donepezil, galantamine, memantine, rivastigmine, or tacrine.
